(19) United States
(12) Patent Application Publication
(10) Pub. No.: US 2025/0051248 A1
(43) Pub. Date: Feb. 13, 2025

(54) REACTOR FOR OXIDATIVE COUPLING OF METHANE

(71) Applicant: SABIC Global Technologies B.V., Bergen op Zoom (NL)

(72) Inventors: Pankaj Singh Gautam, Bengaluru (IN); David West, Bengaluru (IN); Robert Broekhuis, Bengaluru (IN); Tian Gu, Bengaluru (IN)

(21) Appl. No.: 18/720,987

(22) PCT Filed: Dec. 16, 2022
(86) PCT No.: PCT/EP2022/086475

§ 371 Date: Jun. 17, 2024

(30) Foreign Application Priority Data
Dec. 27, 2021 (EP) 21217768.7

(51) Int. Cl. C07C 2/84; B01J 19/00

(52) U.S. Cl. CPC C07C 2/84; B01J 19/0006; B01J 19/0053

(57) ABSTRACT

An oxidative methane coupling (OCM) reactor has a reactor vessel that defines a reactor vessel interior. The reactor vessel having opposite ends and a central longitudinal axis that extends between the opposite ends. A catalyst bed assembly is positioned within the reactor vessel interior having a catalyst bed containing a layer of OCM catalyst of a uniform thickness. The catalyst bed assembly divides the reactor vessel interior into an upstream zone and a downstream zone. The catalyst bed has an upstream face for receiving the one or more flowing feed gases of methane and oxygen gas as a flowing mixture from the upstream zone. The upstream face of the catalyst bed is configured to have a total area that exceeds the largest transverse cross-sectional area of the interior of the reactor vessel that is perpendicular to the central longitudinal axis of the reactor vessel. The catalyst bed assembly is configured so that any portion of the flowing mixture passes from the upstream zone to the downstream zone through a single catalyst bed of the catalyst bed assembly.

### CROSS REFERENCE TO RELATED APPLICATION

This application is a National Stage Application under 35 U.S.C. § 371 and claims the benefit of International Application No. PCT/EP2022/086475, filed on 16 Dec. 2022 and titled ‘Reactor for Oxidative Coupling of Methane’, which application claims priority to EP21217768 filed on 27 Dec. 2021 and titled ‘Reactor for Oxidative Coupling of Methane’. The entire content of both of these applications is incorporated herein by reference.

### TECHNICAL FIELD

The disclosure relates to the conversion of methane and oxygen to C₂ and higher hydrocarbons and reactor designs for such conversion.

### BACKGROUND

Methane can be used to produce ethane and/or ethylene through the oxidative coupling of methane (OCM) reaction. While extensive research and development has been devoted to this reaction, the reaction largely remains inefficient on a commercial scale. One of the key challenges is the high reaction temperature (typically greater than 750° C.) required to make the reaction proceed. The need for such a high temperature is due to the bond strength (bond dissociation energy) of the tetrahedral C—H bonds in methane, which is 104 kcal per mol (kcal/mol). This C—H bond strength makes methane less reactive and difficult to undergo oxidative conversion to form ethylene.

The oxidative coupling of methane reaction can be represented by Equations (1) and (2) below:

2CH₄+O₂→C₂H₄+2H₂O ΔH=−67.4 kcal/mol (1)

2CH₄+½O₂→C₂H₆+H₂O ΔH=−84.6 kcal/mol (2)

As shown in Equations (1) and (2), oxidative conversion of methane to ethylene or ethane is exothermic. Excess heat produced from these reactions can push conversion of methane to carbon monoxide and carbon dioxide rather than the desired C₂ hydrocarbon product, as shown in Equations (3) and (4) below:

CH₄+1½O₂→CO+2H₂O ΔH=−82.8 kcal/mol (3)

CH₄+2O₂→CO₂+2H₂O ΔH=−95.9 kcal/mol (4)

It should be noted that the heats of reaction for Equations (1) to (4) are given per mole of oxygen consumed. The excess heat from the reaction in Equations (3) and (4) further exacerbates this situation, thereby substantially reducing the selectivity of ethylene production when compared with carbon monoxide and carbon dioxide production.

There are two practical problems that have prevented the development of commercially feasible OCM processes. One is the very large heat of reaction (Equations 1-4); and two is the very high temperature to initiate the reaction (typically 700° C.-950° C.). There is no commercially available liquid heat transfer fluid capable of operating at such high temperatures. Consequently, the only way to cool a reactor at this range of temperature is with very inefficient gas phase coolants (e.g., air, steam, ethane, etc.). In a cooled multi-tubular fixed bed reactor, the methane conversion must be limited by the oxygen concentration in the feed to less than about 8% in order to avoid a runaway reaction. A runaway reaction is one in which the temperature rise within the catalyst bed is high enough to damage or deactivate the catalyst or to increase the production of by-products (COₓ).

To overcome these issues, the concept of using adiabatic autothermal reactors (AATRs) for OCM reactions has been developed. Such AATRs and their use in OCM processes are described in U.S. Pat. No. 10,941,088 and U.S. Pub. Pat. App. No. US2021/0031161.

In an AATR, the maximum mass velocity through the catalyst bed that can be achieved relates primarily to catalyst activity and effective catalyst bed thermal conductivity. These parameters also constrain the effective length of the catalyst bed, i.e., the distance traversed through the catalyst bed by the process gas. For optimal catalyst and catalyst bed configurations, the effective length of the catalyst bed is no more than a few inches, while the mass velocities are no greater than 1 kg/m²s. Therefore, to achieve an industrially desired reactor throughput, the surface area of the catalyst bed presented to the feed flow must be sufficiently large. Given practical limits on reactor diameters, and assuming conventional configurations of the catalyst in the reactor, the catalyst bed is limited to the size of the inside diameter of the reactor. This is illustrated in FIG. 1, which shows a conventional disk-like packed-bed of catalyst within a cylindrical reactor vessel, wherein the upstream face of the catalyst bed is perpendicular to the longitudinal axis of the reactor and has a total area that is no greater than the cross-sectional area of the reactor vessel. With such configurations, a world-scale ethylene production facility using the OCM process would require many reactors, resulting in an excessively high capital cost.

The present disclosure overcomes these disadvantages, with the methods and reactors described herein being particularly useful for commercial scale OCM operations that would reduce the number and/or size of the reactors used while increasing the volumetric productivity of the reactor.

### SUMMARY

An oxidative methane coupling (OCM) reactor includes a reactor vessel that defines a reactor vessel interior. The reactor vessel has opposite ends and a central longitudinal axis that extends between the opposite ends. A reactor inlet allows the introduction of one or more flowing feed gases comprising methane and oxygen gas into the interior of the reactor vessel.

A catalyst bed assembly is positioned within the reactor vessel interior. The catalyst bed assembly has at least one catalyst bed containing a layer of OCM catalyst of a uniform thickness. The catalyst bed assembly divides the reactor vessel interior into an upstream zone and a downstream zone. The at least one catalyst bed of the catalyst bed assembly has an upstream face for receiving the one or more flowing feed gases as a flowing mixture from the upstream zone. The upstream face of the at least one catalyst bed of the catalyst bed assembly is configured to have a total area that exceeds the largest transverse cross-sectional area of the interior of the reactor vessel that is perpendicular to the central longitudinal axis of the reactor vessel. The at least one catalyst bed assembly is configured so that any portion of the flowing mixture passes from the upstream zone to the downstream zone through only a single catalyst bed of the catalyst bed assembly. A reactor outlet is located downstream from the catalyst bed assembly that is in fluid communication with the downstream zone for removing reaction products from the reactor.

In certain embodiments, the upstream face is non-perpendicular to the central longitudinal axis of the reactor vessel. In particular applications, the upstream face or a portion of the upstream face of the at least one catalyst bed may be configured as at least one of a cone, a partial cone, a cylinder, a partial cylinder, a cube, a partial cube, a cuboid, a partial cuboid, a sphere, a partial sphere, a spheroid, a partial spheroid, a pyramid, a partial pyramid, a plane that is non-perpendicular to the central longitudinal axis of the reactor vessel, and a non-planar face. In some instances, the upstream face of the at least one catalyst bed is configured as a cylinder or partial cylinder.

The catalyst bed assembly may comprise at least two catalyst beds having upstream faces configured as two or more planes that are parallel to one another in particular cases. The two or more planes may be perpendicular to the central longitudinal axis of the reactor vessel.

The upstream face of the at least one catalyst bed may be configured as a cone or partial cone. In certain applications, the catalyst bed assembly may comprise at least two catalyst beds that are spaced apart along the longitudinal axis of the reactor vessel.

The total area of the upstream face of the at least one catalyst bed may exceed the largest transverse cross-sectional area of the interior of the reactor vessel that is perpendicular to the central longitudinal axis of the reactor vessel by 50% or more.

The layer of OCM catalyst may have a thickness of 50 mm or less. In some cases, the catalyst bed assembly may have only one catalyst bed. The at least one catalyst bed may be a contiguous catalyst bed that has a symmetrical configuration about the central longitudinal axis of the reactor. In some applications, the at least one catalyst bed may be configured with porous channel walls that define longitudinally extending cell channels, the layer of OCM catalyst being positioned along at least a portion of the cell channels, a portion of the cell channels being closed at an upstream end with the downstream end being open to form an outlet of the cell channels, and wherein the remainder of the cell channels are closed at the downstream end and open at the upstream end to form an inlet of the cell channels. The at least one catalyst bed may be configured to have a heat Peclet number (Peh) of from 5 or less.

In a method of carrying out autothermal oxidative coupling of methane (OCM), flowing feed gases comprising methane and oxygen gas are introduced into a reactor as a flowing mixture. The reactor comprises a reactor vessel that defines a reactor vessel interior. The reactor vessel has opposite ends and a central longitudinal axis that extends between the opposite ends. The reactor further comprises a reactor inlet for introducing one or more flowing feed gases comprising methane and oxygen gas into the interior of the reactor vessel. A catalyst bed assembly is positioned within the reactor vessel interior having at least one catalyst bed containing a layer of OCM catalyst of a uniform thickness. The catalyst bed assembly divides the reactor vessel interior into an upstream zone and a downstream zone. The at least one catalyst bed of the catalyst bed assembly has an upstream face for receiving the flowing mixture from the upstream zone. The upstream face of the at least one catalyst bed of the catalyst bed assembly is configured to have a total area that exceeds the largest transverse cross-sectional area of the interior of the reactor vessel that is perpendicular to the central longitudinal axis of the reactor vessel. A reactor outlet located downstream from the catalyst bed assembly is in fluid communication with the downstream zone for removing reaction products from the reactor.

In the method, the flowing mixture is allowed to pass from the upstream zone to the downstream zone so that any portion of the flowing mixture flows through only a single catalyst bed of the catalyst bed assembly, and wherein methane and oxygen gas of the flowing mixture contact the OCM catalyst of the at least one catalyst bed to react to form methane oxidative coupling reaction products that are received in the downstream zone. The reaction products are removed from the downstream zone through the reactor outlet.

### BRIEF DESCRIPTION OF THE DRAWINGS

For a more complete understanding of the embodiments described herein, and the advantages thereof, reference is now made to the following descriptions taken in conjunction with the accompanying figures, in which:

FIG. 1 is a front perspective view of a prior art reactor shown in cross section that employs a flat, disk-like packed-bed of catalyst within a cylindrical reactor vessel with the catalyst bed oriented perpendicular to the longitudinal axis of the reactor vessel in a conventional manner; and

FIG. 2 is an elevational view of an OCM reactor shown in cross section employing an annular catalyst bed constructed in accordance with particular embodiments of the disclosure;

FIG. 3 is a transverse cross-sectional view of the OCM reactor of FIG. 2 taken along the lines 3-3;

FIG. 4 is a perspective view of an OCM reactor oriented horizontally, which includes a planar catalyst bed that is centered on and is parallel with a central longitudinal axis of the reactor vessel and is constructed in accordance with particular embodiments of the disclosure;

FIG. 5 is a transverse cross-sectional view of the OCM reactor of FIG. 4 taken along the lines 5-5;

FIG. 6 is a transverse cross-sectional view of an OCM reactor employing two planar catalyst beds that are parallel to one another and to a central longitudinal axis of the reactor vessel, with the catalyst bed being spaced apart on either side of the longitudinal axis of the reactor vessel, and constructed in accordance with particular embodiments of the disclosure;

FIG. 7 is an elevational view of an OCM reactor shown in cross section employing a frustoconical reactor bed and constructed in accordance with particular embodiments of the disclosure;

FIG. 8 is an elevational view of an OCM reactor shown in cross section employing multiple stacked conical reactor beds and constructed in accordance with particular embodiments of the disclosure;

FIG. 9 is an elevational view of an OCM reactor shown in cross section that employs multiple flat disk-shaped reactor beds that are each perpendicular to a longitudinal axis of the reactor and constructed in accordance with particular embodiments of the disclosure;

FIG. 10 is an elevational view of an OCM reactor shown in cross section employing a meandering or undulating reactor bed having various peaks and valleys and constructed in accordance with particular embodiments of the disclosure;

FIG. 11 is an elevational view of an OCM reactor shown in cross section employing reactor bed having a honeycomb-type structure and constructed in accordance with particular embodiments of the disclosure;

FIG. 12 is a top perspective view of the catalyst bed of the OCM reactor of FIG. 11; and

FIG. 13 is an enlarged elevational cross-sectional view of a cell channel and channel walls of the catalyst bed of FIG. 11.

### DETAILED DESCRIPTION

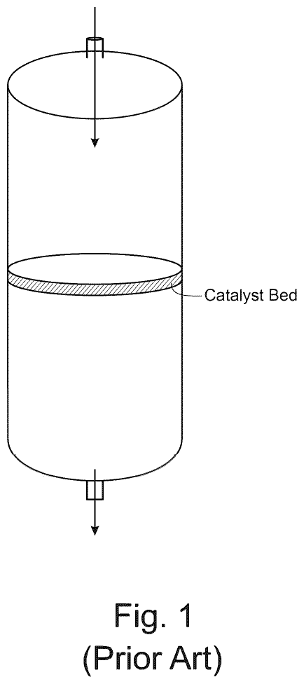

As discussed in the background section, the key to increasing volumetric productivity of the adiabatic autothermal reactor (AATR) in OCM reactions is to increase the area of the inflow or upstream face of the catalyst bed to the incoming reactor feed. In the present disclosure, this is achieved by arranging the inflow or upstream face of the catalyst bed in a way that is different from the flat, perpendicular, circular geometry of the conventional single catalyst beds, such as those in FIG. 1. Because the desired OCM reaction products (i.e., ethane and ethylene) are themselves prone to being further converted to non-desired byproducts when placed in contact with OCM catalysts, the reactor and catalyst bed or beds must be configured so the process fluids pass or traverse through only one catalyst bed without passing again through any catalyst bed before exiting the reactor. The unique reactor and catalyst bed designs of the disclosure and their operation make it possible to obtain higher volumetric productivity and higher yields of OCM reaction products (e.g., C₂ hydrocarbons) than can be obtained with conventional adiabatic reactors.

The particular reactors and catalyst beds described herein are configured for and operated so that adiabatic or near-adiabatic auto-thermal oxidative coupling of methane can be achieved. While adiabatic conditions are desired, in practice only near-adiabatic conditions can be maintained. This is true even though reference is commonly made to the use of adiabatic reactors. Those skilled in the art will recognize that there is some heat transfer in such adiabatic reactors so they are not perfectly adiabatic. Accordingly, the use of the term “adiabatic” throughout this disclosure may therefore refer to such near-adiabatic conditions, which can be defined as from 10% or 5% or less heat transfer or heat loss from the reactor (relative to the total heat generated). Furthermore, as used herein, the expression “auto-thermal” with respect to the OCM reaction described means that only the heat produced by the reaction itself is used to carry out the reaction. This means that once the reaction commences within the reactive zone, where the reactive zone constitutes those areas downstream of the upstream face of the catalyst bed, no heating from other sources is provided to carry out the oxidative coupling reaction when it reaches steady state.

It should be noted in the description, if a numerical value, concentration or range is presented, each numerical value should be read once as modified by the term “about” (unless already expressly so modified), and then read again as not so modified unless otherwise indicated in context. Also, in the description, it should be understood that an amount range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific points within the range, or even no point within the range, are explicitly identified or referred to, it is to be understood that the inventor appreciates and understands that any and all points within the range are to be considered to have been specified, and that inventor possesses the entire range and all points within the range.

In the following discussion, various configured catalyst beds for oxidative methane coupling (OCM) reactions are described. While certain configurations are shown and described for illustrative purposes, it should be apparent that variations and combinations of the various catalyst beds described can be used alone or in combination with one another. Non-limiting configurations of the catalyst beds are those catalyst beds having an upstream face or a portion of the upstream face that may be configured as a cone, a partial cone, a cylinder, a partial cylinder, a cube, a partial cube, a cuboid, a partial cuboid, a sphere, a partial sphere, a spheroid, a partial spheroid, a pyramid, a partial pyramid, a plane that is non-perpendicular to the central longitudinal axis of the reactor vessel, and a non-planar face.

In each of the catalyst bed configurations, the catalyst bed(s) is (are) configured to have an upstream face or faces with a total area that exceeds the largest transverse cross-sectional area of the interior of the reactor vessel that is perpendicular to the central longitudinal axis of the reactor vessel. The catalyst bed(s) is (are) configured so that each flow of the flowing mixture passes through only one catalyst bed without passing through any other catalyst bed.

As will be described in more detail later, the catalyst beds are configured to meet certain requirements related to the dimensionless Peclet (Pe) numbers. The catalyst beds are configured to have a rather large area but a relatively shallow depth or thickness. As used herein with reference to the various catalyst beds described herein and their components, the terms “thickness,” “depth,” and the like, as it refers to the catalyst beds refers to the linear distance as measured between the opposite upstream and downstream faces of the catalyst bed. In certain embodiments, the catalyst bed thickness may be 50 mm or less. In particular instances, the catalyst bed thickness may be at least, equal to, and/or between any two of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, and 40 mm, 41 mm, 42 mm, 43 mm, 44 mm, 45 mm, 46 mm, 47 mm, 48 mm, 49 mm, and 50 mm.

The catalyst used for the catalyst beds described herein may not be limited to any particular type of catalyst provided it is suitable for the OCM reaction and facilitates meeting the necessary requirements for the catalyst bed configuration and operation, as is described in more detail later on. The OCM catalyst should have a high enough activity to provide the desired OCM conversion with the high space velocities or space time used and reaction conditions, as described herein. One or more different OCM catalysts can be used.

The catalysts may be supported catalysts, bulk metal catalysts, and/or unsupported catalysts, or combinations of these. The support can be active or inactive. The catalyst support can include MgO, Al₂O₃, SiO₂, or the like. All the support materials are those currently available or that can be formed from those processes known in the art. These may include precipitation/co-precipitation, sol-gel, templates/surface derivatized metal oxides synthesis, solid-state synthesis, of mixed metal oxides, microemulsion techniques, solvothermal, sonochemical, combustion synthesis, etc.

One or more of the catalysts can include one or more metals or metal compounds thereof. Non-limiting catalytic metals include Li, Na, Ca, Cs, Mg, La, Ce, W, Mn, Ru, Rh, Ni, Pt, Nd, Pr, and Tb and combinations and alloys of these. Non-limiting examples of suitable catalysts include: (1) La on a MgO support; (2) Na, Mn, and La₂O₃ on an aluminum support; (3) Na and Mn on a silicon dioxide support; (4) Na₂WO₄ and Mn on a silicon dioxide support, and combinations of these. Non-limiting examples of some particular catalysts that can be used include La₂CeO₂, SrO/La₂O₃, CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, W₂O₃, SnO₂, Yb₂O₃, Sm₂O₃, SrO/La₂O₃, La₂O₃, Ce₂O₃, La/MgO, oxides of other rare earth metals, and combinations thereof.

The catalyst of the catalyst beds may take several different forms. In one form, this may include a shallow layer of catalyst particles having particle sizes of from 0.1 mm to 10 mm, more particularly from 0.1 mm, 0.2 mm, 0.3 mm, 0.4 mm, 0.5 mm, 0.6 mm, 0.7 mm, 0.8 mm, 0.9 mm, or 1 mm to 2 mm, 3 mm, 4 mm, 5 mm, 6 mm, 7 mm, 8 mm, 9 mm, or 10 mm. In certain embodiments the catalyst particles making up the shallow layer may have particle size ranging from 0.1 mm to 3 mm, 4 mm, or 5 mm. The catalyst particles making up the catalyst bed may be of the same or different sizes. The catalyst particles may rest on or in a catalyst bed supporting structure or enclosure. Such support or enclosure may include, but is not limited to a perforated ceramic support plate, quartz fiber mats, stainless steel screens, stainless steel coated with fused silica, etc. The catalyst particles can be of eggshell type or constitute a coating on a high conductivity non-catalytic particle. The catalyst bed can be a mix of OCM catalyst and non-catalytic high conductivity particles.

In another form, the catalyst beds may be composed of one or more porous monolithic bodies. The monolithic body may be a ceramic or metal material having pores or channels with a pore or channel size (i.e., the transverse width or diameter) of from 0.1 mm to 5 mm, more particularly from 0.5 mm to 2.0 mm. The thickness of the monolithic bodies forming the catalyst bed 20 may range from 5 mm to 50 mm. In particular embodiments, the thickness of the monolithic bodies may be at least, equal to, and/or between any two of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, and 40 mm, 41 mm, 42 mm, 43 mm, 44 mm, 45 mm, 46 mm, 47 mm, 48 mm, 49 mm, and 50 mm. All or a portion of the pore channels of the monolithic body may be oriented parallel to the central axis or direction of flow through the reactor 10. In other embodiments, the pores may be non-parallel to the direction of flow or may be randomly oriented, such as a ceramic or metal foam material. The pores should be continuous or contiguous through the thickness of the monolithic body to allow passage of gases therethrough from the upstream face to the downstream face of the catalyst bed. The cross-sectional shape of the pores may vary but in particular embodiments may be circular, oval, square, rectangular, polygonal, etc. In other embodiments, all or a portion of the cross-sectional shape of the pores may be irregular or non-uniform in shape.

The monolithic body or bodies are either formed from or are provided with an OCM catalyst material present on all or a portion of the surfaces of the monolithic body. In particular, at least all or a portion of the surfaces of the pore channels are coated with such OCM catalyst material, such as those OCM catalyst material described previously. The amount of OCM catalyst material provided on the monolithic bodies is that sufficient to carry out the OCM reaction, as described herein.

In still another form, the catalyst beds may be composed of one or more porous monolithic bodies similar to those described above. The pore sizes may be the same as those previously described, i.e., pore or channel size (i.e., the transverse width or diameter) of from 2 mm to 10 mm. In certain instances, the pore or channel size may be at least, equal to, and/or between any two 2 mm, 3 mm, 4 mm, 5 mm, 6 mm, 7 mm, 8 mm, 9 mm, and 10 mm. The thickness of the monolithic bodies forming the catalyst bed may range from 5 mm to 50 mm. In particular embodiments, the thickness of the monolithic bodies thickness of the monolithic bodies may be at least, equal to, and/or between any two of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, and 40 mm, 41 mm, 42 mm, 43 mm, 44 mm, 45 mm, 46 mm, 47 mm, 48 mm, 49 mm, and 50 mm. Unlike the monolithic bodies previously described, the surfaces of the monolithic bodies may not be coated or provided with an OCM catalytic material, although in certain instances they may be coated with such an OCM catalytic material, as well. Instead, all or a portion of the pore channels of the monolithic body or bodies are filled with OCM catalyst particles or powder, such as the OCM catalyst particle materials described previously. The OCM catalyst particles or powder may have a particle size of from 1000 microns, 900 microns, 800 microns, 700 microns, 600 microns, 500 microns, 400 microns, 300 microns, 200 microns, 100 microns or less. All or a portion of the pores of the monolithic are filled with the OCM catalyst particles or powder to carry out the OCM reaction.

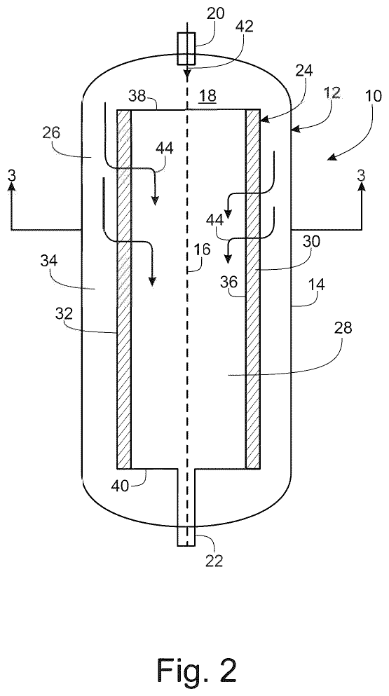

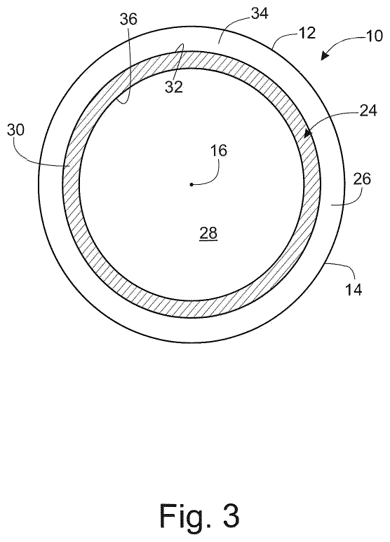

Referring now to specific examples of reactors that employ the unique catalyst bed configurations, FIGS. 2 and 3 illustrate an exemplary reactor 10 employing a novel catalyst bed and in which the OCM reaction may be carried out. The reactor 10 includes a reactor vessel 12 having a cylindrical reactor wall 14 that is coaxial with a central longitudinal axis 16 of the reactor vessel 12. In other embodiments, the reactor wall 14 may be non-cylindrical along all or a portion of its length, but otherwise have rotational symmetry about the central longitudinal axis 16 or be of varying diameter along all or portions of its length, such as spherical, spheroidal, conical, frustoconical, etc.

The reactor vessel 12 defines an interior 18 of the reactor 10. One or more inlets 20 of the reactor vessel 12 are provided for introducing one or more flowing feed gases that comprise methane and oxygen gas (O₂) into the interior of the reactor vessel 12. The reactor 10 and/or the catalyst bed (i.e., the reaction zone) may be configured as an adiabatic reactor or catalyst bed (or near-adiabatic) to prevent or reduce heat transfer or loss through the walls of the reactor vessel 12. Typically, the reactor 10 will be oriented for vertical flow, with the inlet 20 being located on the top or bottom of the reactor vessel 12. In the embodiment shown, the inlet 20 is located at the top of the reactor vessel 12 so that fluid flow is directed downward through the reactor 10. An outlet 22 at the bottom of the reactor vessel 12 is provided to remove reaction products from the interior of the reactor vessel 12.

In the following discussion, the reactor vessels generally may have the same or a similar configuration. Accordingly, the same reference numerals may be used throughout the discussion with respect to the reactor vessels and those components that are common to the reactors for each of the different reactors and catalyst bed configurations described.

An annular catalyst bed assembly 24 is positioned within the interior 18 of the reactor vessel 12 and divides the interior 18 of the vessel 12 into an inlet or upstream zone 26 and an outlet or downstream zone 28. The catalyst bed assembly 24 includes a catalyst bed 30 that constitutes a layer of catalyst material suitable for carrying out the OCM reaction or similar reactions, such as those previously described. The catalyst bed 30 is configured as cylinder that is concentric with the longitudinal axis 16 and cylindrical wall 14 of the reactor vessel 12. The catalyst bed 30 may be contiguous and have a symmetrical configuration about the central longitudinal axis 16 of the reactor 10. The catalyst bed 30 extends longitudinally along all or a portion of the length of cylindrical reactor wall 14. An upstream face 32 of the catalyst bed 30 interfaces with the upstream zone 26 of the reactor vessel 12 and is radially spaced a distance inward around its circumference from the interior surface of the reactor wall 14 to provide an annular gap or flow space 34 between the catalyst bed 30 and the interior of the reactor wall 14. The flow space 34 forms a part of the upstream zone 26 of the reactor vessel 12 and is in fluid communication with the reactor inlet 20. A downstream face 36 of the catalyst bed 30 that interfaces with the downstream zone 28 is radially spaced apart from the longitudinal axis 16 around its circumference.

The flow space 34 is sized and configured so that it does not impede gas flow within the upstream zone 26 as it flows from the inlet 20 through the annular space 34 of the upstream zone 24 along the entire longitudinal length of the catalyst bed 30. Both the flow space 34 and catalyst bed 30, as well as other areas of the upstream zone 24, should be configured so the flow velocities through the thickness of the catalyst bed 30 are generally uniform and there is no significant differences in the pressure drop across the thickness of the catalyst bed over all areas of the catalyst bed 30.

If necessary, catalyst bed assembly 24 may include a screen or permeable enclosure that contains the catalyst material of the catalyst bed 30 and allows radial passage of gases from the annular space 34 through the catalyst bed 30 to the outlet or downstream zone 28.

The catalyst bed assembly 24 may include fluid impermeable upper and lower walls or closure structures 38, 40, located at opposite longitudinal ends of the catalyst bed 30 to isolate the downstream zone 28 from the upstream zone 26 so that all gases or fluids must pass through the annular catalyst bed 30 from the upstream zone 26 to the downstream zone 28. In an alternate arrangement of the embodiment of FIGS. 1 and 2, all or a portion of either of the walls or closure structures 38, 40 may instead be formed as part of the catalyst bed 30, as well, with the walls or closure structures 38, 40 being formed as layers of catalyst bed material through which the feed gases flow. In other embodiments, the catalyst bed 30 may extend the full length of the reactor vessel 12, with the ends of the reactor vessel 12 forming the closure structures at each end of the catalyst bed 30. In such cases, the inlet 20 may be configured to communicate directly with the annular space 34.

The outlet 22 of the reactor 10 is located downstream from the catalyst bed 30 and is in fluid communication with the downstream zone 28 for removing OCM reaction products from the reactor vessel 12. In particular applications, the reactor 10 is configured so that the overall pressure drop from the reactor inlet 20 to the reactor outlet 22 is from 0.5 bar or less.

In use, the reactor 10 may be operated as an AATR at conditions necessary to carry out the methane oxidative coupling reactions described herein. Feed gas 42 from reactor inlet 20 flows as a flowing mixture comprising methane and oxygen gas within the upstream zone 26 of the reactor vessel interior 18. The feed gas mixture fills the entire volume of the upstream zone 26 of the reactor vessel 12 and passes as gas flows, indicated by the arrows 44, through the catalyst bed thickness from the upstream face 32 of the catalyst bed 30 to the downstream face 36 of the catalyst bed 30 to the downstream zone 28. The feed gas mixture flows so that each portion or flow 44 of the flowing mixture passes only once through the catalyst bed 30 to the downstream zone 28 without passing through any other area of the catalyst bed 30 a second or further time. As the flowing mixture flows through the OCM catalyst of the catalyst bed 30, the methane and oxygen of the flowing mixture reacts to form methane oxidative coupling reaction products, such as ethane and ethylene, which flow as effluent from the downstream face 36 of the catalyst bed 30 to the downstream zone 28.

The reaction products from downstream zone 28 are then discharged and removed from the reactor 10 through reactor outlet 22. The reaction products may be collected and stored or directed to other processing equipment for further processing. This may include cracking and/or quenching of the reaction products, which may be carried out in other vessels or equipment external to the reactor 10 or reactor vessel 12 for such purposes.

In certain applications of the embodiment of the reactor 10 shown in FIGS. 2 and 3, the interior space of the downstream zone 28 within the catalyst bed 30 can be configured and designed to include adiabatic thermal pyrolysis conversion of the effluent or reaction products from the catalyst bed 28.

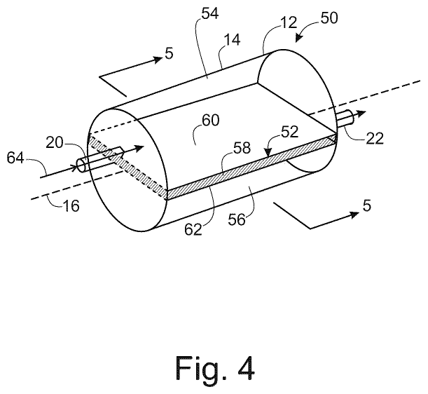

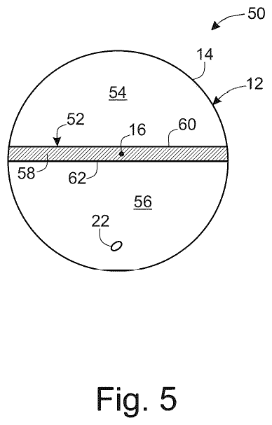

Referring to FIGS. 4 and 5, another example of a reactor 50 is shown employing a novel catalyst bed in which the OCM reaction may be carried out. The reactor 50 functions similarly to the reactor 10, previously described. The same or similar components of the reactor vessel are labeled with the same reference numerals. The reactor 50 includes a reactor vessel 12 having a cylindrical reactor wall 14 that is coaxial with a central longitudinal axis 16 of the reactor vessel 12. In other embodiments, the reactor wall 14 may be non-cylindrical along all or a portion of its length, but may otherwise have rotational symmetry about the central longitudinal axis 16 or be of varying diameter along all or portions of its length, such as spherical, spheroidal, conical, frustoconical, etc. As is shown in FIG. 4, the reactor vessel 12 is typically oriented horizontally, i.e., with the central longitudinal axis 16 being horizontal. In other embodiments, the vessel 12 can be oriented vertically or at an angle between horizontal and vertical.

The reactor vessel 12 defines an interior 18 of the reactor 50. The reactor 50 may be configured as an adiabatic reactor (or near-adiabatic) to prevent or reduce heat transfer or loss through the walls of the reactor vessel 12. A catalyst bed assembly 52 is positioned within the interior of the reactor vessel 52 and extends across the width of the reactor vessel 52 to divide the interior of the vessel 52 into an inlet or upstream zone 54 and an outlet or downstream zone 56. One or more inlets 20 of the reactor vessel 12 are provided that are in fluid communication with the upstream zone 54 for introducing one or more flowing feed gases that comprise methane and oxygen gas (O₂) into the upstream zone 54.

The catalyst bed assembly 52 is constructed with a planar catalyst bed 58 that constitutes a flat layer of catalyst material configured as a thin body having opposite upstream and downstream faces 60, 62 (FIG. 5), respectively, that are parallel to one another. In the embodiment shown, the faces 60, 62 are parallel to the longitudinal axis 56 with the central axis 56 passing through the center of the catalyst bed 58. In other instances, the catalyst bed 58 may be offset from the axis 56, such as to one side of the axis 56, and the faces 60, 62 may be parallel or non-parallel with the axis 56.

The catalyst bed 58 may extend along all or a portion of the length of cylindrical reactor wall 54. The upstream face 60 of the catalyst bed 58 interfaces with the upstream zone 54 of the reactor vessel 52, with the upstream zone 54 constituting a flow space between the catalyst bed 58 and the interior of the reactor wall 54. The upstream zone 54 of the reactor vessel 52 is in fluid communication with the reactor inlet 58.

Both the catalyst bed 58 and the upstream zone 54 should be configured so the flow velocities through the thickness of the catalyst bed 58 are generally uniform and there are no significant differences in the pressure drop across the thickness of the catalyst bed 58 over all areas of the catalyst bed 58.

The catalyst bed assembly 52 may include a screen or permeable enclosure that contains the catalyst material of the catalyst bed 58 to allow the passage of gases from the upstream zone 54 through the catalyst bed 58 to the downstream zone 56.

The ends of the reactor vessel 52 are closed off with a reactor outlet 22 of the reactor vessel 12 being in fluid communication with the downstream zone 56 for removing effluent and reaction products from the downstream zone 56 of the reactor 50.

In use, the reactor 50 may be operated as an AATR at conditions necessary to carry out the methane oxidative coupling reactions described herein. Feed gas 64 from reactor inlet 20 flows as a flowing mixture comprising methane and oxygen gas within the upstream zone 54 of the interior of the reactor vessel 52. The feed gas mixture fills the entire volume of the upstream zone 54 of the reactor vessel 52, with the feed gas mixture flowing through the catalyst bed thickness from the upstream face 60 to the downstream face 62 of the catalyst bed 58 to the downstream zone 62. The feed gas mixture flows so that portions or flows of the flowing mixture passes only once through the catalyst bed 58 to the downstream zone 56 without passing again through any other area of the catalyst bed 58 a second time.

As the flowing mixture flows through the OCM catalyst of the catalyst bed 58, the methane and oxygen of the flowing mixture reacts to form methane oxidative coupling reaction products, such as ethane and ethylene, that flow as effluent from the downstream face 62 of the catalyst bed 58 to the downstream zone 56. The reaction products from downstream zone 56 are then discharged and removed from the reactor 50 through reactor outlet 22 for further processing and/or storage.

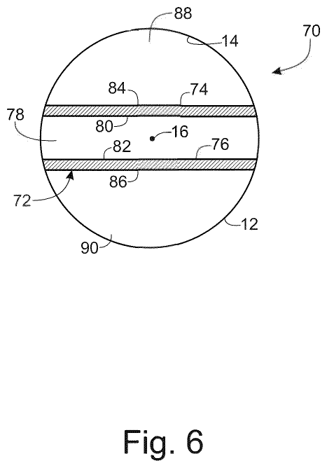

Referring to FIG. 6, another embodiment of a reactor 70 is shown. The reactor 70 is similar to the reactor 50, previously described, with similar components of the reactor vessel labeled with the same reference numerals. The reactor 70 differs from the reactor 50 in that it employs catalyst bed assembly 72 having two catalyst beds 74, 76. The catalyst beds 74, 76 are also thin, planar catalyst beds, similar to the catalyst bed 58 of FIGS. 4 and 5. The beds 74, 76 are parallel to one another and to the longitudinal axis 16 of the reactor vessel 12. The catalyst beds 74, 76 are each positioned on opposite sides of the axis 16, however, with the area between the beds 74, 76 constituting an upstream zone 78 that is in fluid communication with the inlet 20 of the reactor 70. The inward facing faces 80, 82 of the catalyst beds 74, 76, respectively, form the upstream faces of the catalyst beds 74, 76, while the opposite faces 84, 76 of the catalyst beds 74, 76 form the downstream faces. The areas between the downstream faces 84, 86 and the interior of the reactor vessel wall 54 form downstream zones 88, 90 of the reactor 70 that are in fluid communication with reactor outlets for each zone 88, 90, such as outlet 22.

The operation of reactor 70 is similar to that of reactor 50, but with the feed gas mixture from the reactor inlet 20 flowing between the catalyst beds 74, 76 in the upstream zone 78. The feed gas mixture flows so that portions or flows of the flowing mixture passes only once through one of the catalyst beds 74, 76 to one of the downstream zones 88, 90 without passing through any other area of any catalyst bed a second time. As the flowing mixture flows through the OCM catalyst of the catalyst beds 74, 76 the methane and oxygen gas react to form OCM reaction products. The reaction products from downstream zones 88, 90 are then discharged and removed from the reactor 70 through the reactor outlet 22. Given the same reactor catalyst bed dimensions to that of reactor 50 of FIGS. 4 and 5, the reactor 70 employing two catalyst beds 74, 76 doubles the total area for the upstream faces for the catalyst bed from that of reactor 50.

In a variation of the reactor 70, more than two or a plurality of catalyst beds, such as the catalyst beds 74, 76, can be positioned within a single reactor vessel to provide multiple upstream and downstream zones, which are in fluid communication with the reactor inlet(s) and outlet(s), respectively, to increase the total area of the catalyst beds within the same reactor.

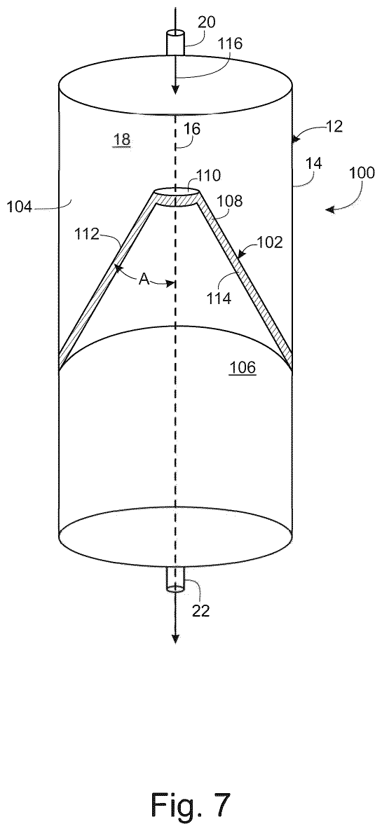

FIG. 7 shows a further embodiment of a reactor 100 that employs a novel catalyst bed in which the OCM reaction may be carried out. The reactor 100 includes a reactor vessel 12 having a cylindrical reactor wall 14 that is coaxial with a central longitudinal axis 16 of the reactor vessel 12. In other embodiments, the reactor wall 14 may be non-cylindrical along all or a portion of its length, but otherwise have rotational symmetry about the central longitudinal axis 16 or be of varying diameter along all or portions of its length, such as spherical, spheroidal, conical, frustoconical, etc.

The reactor vessel 12 defines an interior 18 of the reactor 100. One or more inlets 20 of the reactor vessel 12 are provided for introducing one or more flowing feed gases that comprise methane and oxygen gas (O₂) into the interior of the reactor vessel 12. The reactor 100 is configured as an adiabatic reactor (or near-adiabatic) to prevent or reduce heat transfer or loss through the walls of the reactor vessel 12. Typically, the reactor 100 will be oriented for vertical flow, with the inlet 20 being located on the top or bottom of the reactor vessel 12. In the embodiment shown, the inlet 20 is located at the top of the reactor vessel 12 so that fluid flow is directed downward through the reactor 100.

A conical or frustoconical catalyst bed assembly 102 is positioned within the interior 18 of the reactor vessel 12 and divides the interior 18 of the vessel 12 into an upstream zone 104 and an outlet or downstream zone 106. The catalyst bed assembly 102 includes a catalyst bed 108 that constitutes a layer of catalyst material suitable for carrying out the OCM reaction or similar reactions as described herein. The catalyst bed 108 is formed in a conical or frustoconical configuration that is concentric with the longitudinal axis 16 and cylindrical wall 14 of the reactor vessel 12. The catalyst bed 108 may be contiguous and have a symmetrically configuration about the central longitudinal axis 16 of the reactor 10.

The widest portion or base of the conical catalyst bed 108 extends radially outward so that it contacts or engages the cylindrical reactor wall 14 around its circumference to isolate the upstream zone 104 from the downstream zone 106. In the embodiment shown, the peak or frustum 110 of the conical catalyst bed 108 is oriented so that it points towards the upstream zone 104 or inlet 20. In other embodiments, however, the conical catalyst bed 108 may be inverted so that the peak or frustum 110 points to the downstream zone 106.

The inlet or upstream face 112 of the catalyst bed 108 interfaces with the upstream zone 104 of the reactor vessel 12, with the opposite downstream face 114 of the catalyst bed 108 interfacing with the downstream zone 106. The conical upstream face 112, as well as the downstream face 114, may be oriented at an angle A relative to the longitudinal axis 16. The angle A may range from greater than 0° to 120°. In certain instances, the angle A may be at least, equal to, and/or between any two of 5°, 10°, 15°, 20°, 25°, 30°, 35° 40°, 45° 50°, 55° 60°, 65°, 70°, 75° 80°, 85°, 90°, 95° 100°, 110°, 115°, and 120°.

The catalyst bed assembly 102 may include a screen or permeable enclosure that contains the catalyst material of the catalyst bed 108 and allows passage of gases from the upstream zone 104 through the catalyst bed 108 to the downstream zone 106.

An outlet 22 of the reactor 100 located downstream from the catalyst bed 108 that is in fluid communication with the downstream zone 106 is provided for removing OCM reaction products 120 from the reactor vessel 12.

In use, the reactor 100 may be operated as an AATR at conditions necessary to carry out the methane oxidative coupling reactions described herein. The feed gas mixture 116 from reactor inlet 20 flows as a flowing mixture comprising methane an oxygen gas within the upstream zone 104 of the interior of the reactor vessel 12. The feed gas mixture fills the entire volume of the upstream zone 104 of the reactor vessel 12, with the feed gas mixture flowing through the thickness of the catalyst bed 108 from the upstream face 112 to the downstream face 114 to the downstream zone 106. All portions of the feed gas mixture pass only once through the catalyst bed 108 to the downstream zone 106 without passing through any area of the catalyst bed 108 a second time.

As the flowing mixture flows through the OCM catalyst of the catalyst bed 108, the methane and oxygen gas of the flowing mixture reacts to form OCM reaction products, such as ethane and ethylene, that flow as effluent from the downstream face 114 of the catalyst bed 108 to the downstream zone 106. The reaction products from downstream zone 106 are then discharged and removed from the reactor 100 through reactor outlet 22.

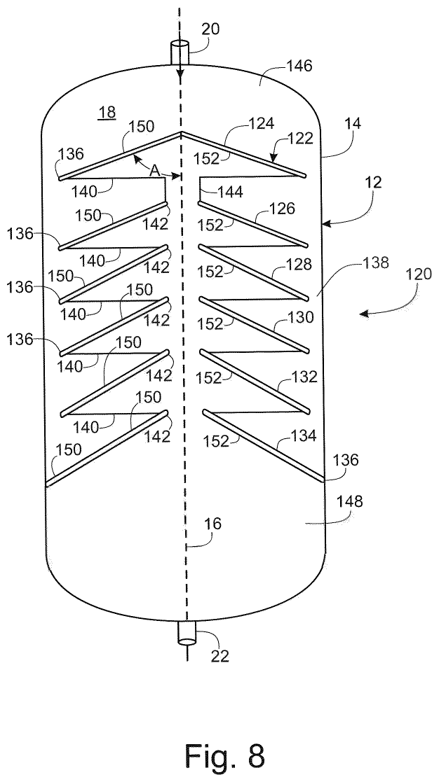

Referring to FIG. 8, a further reactor 120 is shown that is similar to the reactor 100, previously described. The reactor 120 differs from that of reactor 100 in that it incorporates a catalyst bed assembly that utilizes a plurality of conical or frustoconical catalyst beds. As shown, the catalyst bed assembly 122 comprises multiple conical or frustoconical catalyst beds 124, 126, 128, 130, 132, and 134 that are arranged or spaced in a stacked arrangement along the longitudinal axis 16 of the reactor vessel 14. The catalyst bed 124 constitutes the uppermost catalyst bed and the catalyst bed 134 constitutes the lowermost catalyst bed, with catalyst beds 126, 128, 130, and 132 being intermediate catalyst beds positioned between the catalyst beds 124 and 134.

Each of the catalyst beds 124, 126, 128, 130, 132, and 134 may have the same or a similar configuration to the catalyst bed 108 of FIG. 7. The size and shape of each catalyst bed 124, 126, 128, 130, 132, and 134 may be the same or different from the others. The uppermost catalyst beds 124, 126, 128, 130, and 132 are configured so that at all or a portion of the side edges 136 around the base of each conical or frustoconical catalyst bed are spaced radially inward from the interior surface of the reactor vessel wall 14 to define a longitudinal flow space 138. The lowermost catalyst bed 134 is shown having abase wherein the side edges 136 extend radially outward so that they contact or engage the cylindrical reactor wall 14 around its entire circumference.

The catalyst bed assembly 122 may further include fluid impermeable walls, closures or structures 140. The closures 140 extend radially inward from the side edges 136 and circumferentially about the base of each catalyst bed 124, 126, 128, 130, and 132. The closures 140 of each of the intermediate catalyst beds 126, 128, 130, and 132 extend radially inward to openings 142 formed in the peak or frustum of the next lower adjacent conical catalyst bed 128, 130, 132, 134. The closure 140 of the uppermost conical bed 124 extends radially inward to a longitudinal conduit 144 that is in fluid communication with the opening 142 of the lower adjacent catalyst bed 126. In an alternate variation, the conduit 144 may be eliminated from the uppermost catalyst bed 124, with the closure 140 of catalyst bed extending to the opening 142 of catalyst bed 126, as with the remaining catalyst beds. Alternatively, additional conduits, such as the conduit 144 may be used with each catalyst bed. In another arrangement of the catalyst bed assembly 122 of FIG. 8, all or a portion of the walls or closures 140 and/or the conduit 144, instead of being fluid impermeable, may be formed from a layer of the catalyst bed material through which the feed gases flow.

The catalyst bed assembly 122 formed by the catalyst beds 124, 126, 128, 130, 132, 134, their closures 140, the conduit 144 and the lowermost catalyst bed 134 with the side edges 140 engaging the cylindrical reactor wall 14 divide the interior space 18 into an upstream zone 146 and a downstream zone 148. The upper face 150 of each of the catalyst beds 124, 126, 128, 130, 132, 134 forms an upstream face that interfaces with the upstream zone 146 and the lower face 150 of each catalyst bed forms a downstream face that interfaces with the downstream zone 148. The angle A of the conical or frustoconical upper and lower faces 150, 152 of each of the catalyst beds may be the same or different from the others. Because multiple catalyst beds are used in the catalyst assembly 122, the upstream faces 150, as well as the downstream faces 152, may have a more gradual slope in certain embodiments, with the angle A relative to the longitudinal axis 16 ranging from greater than 0° to less than 180°. In many applications, the angle A may range from 20° to 160°, more particularly from 40° to 120°. In certain instances, the angle A may be at least, equal to, and/or between any two of 5°, 10°, 15°, 20°, 25°, 30°, 35° 40°, 45° 50°, 55° 60°, 65°, 70°, 75° 80°, 85°, 90°, 95° 100°, 110°, 115°, 120°, 125°, 130°, 135°, 140°, 145°, 150°, 155°, 160°, 165°, 170°, and 175°.

The configuration of the catalyst bed assembly 122 ensures that all gases or fluids must pass from the upstream zone 146 through the catalyst beds 124, 126, 128, 130, 132, 134 to the downstream zone 28. The openings 142 of the catalyst beds and the conduit 144 provide passages within the downstream zone 148 for flowing gases to reach the reactor outlet 22.

The operation of the reactor 120 is similar to that of the reactors previously described. The reactor 120 may be operated as an AATR at conditions necessary to carry out the methane oxidative coupling reactions described herein. As the flowing mixture flows through the OCM catalyst of the catalyst beds 124, 126, 128, 130, 132, 134, the methane and oxygen of the flowing mixture reacts to form methane oxidative coupling reaction products, such as ethane and ethylene, that flow as effluent from the downstream faces 150 of the catalyst beds to the downstream zone 148. The reaction products from downstream zone 148 are then discharged and removed from the reactor 120 through reactor outlet 22. All portions of the feed gas mixture pass only once through a catalyst bed 124, 126, 128, 130, 132, 134 to the downstream zone 106 without passing through any of the catalyst beds 124, 126, 128, 130, 132, 134 a second time.

The reactor 120 employing multiple conical or frustoconical catalyst beds 124, 126, 128, 130, 132, 134 greatly increases the total area of the upstream faces or catalyst beds from that, such as reactor 100, that only utilizes a single conical or frustoconical catalyst bed.

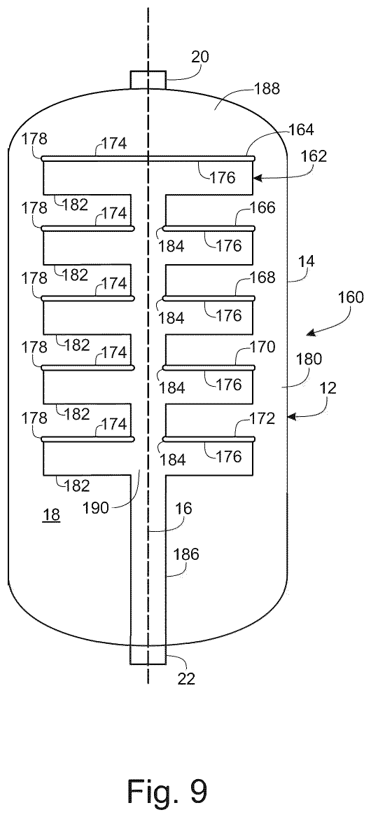

FIG. 9 shows still another embodiment of a reactor 160. The reactor 160 is similar to the reactor 120 utilizing multiple conical or frustoconical catalyst beds, as previously described, with similar components labeled with the same reference numerals. Instead of employing multiple conical or frustoconical catalyst beds, the reactor 160 includes a catalyst bed assembly 162 that employs multiple flat, disk-shaped catalyst beds 164, 166, 168, 170, 172 that are arranged or spaced apart in a stacked arrangement along the longitudinal axis 16 of the reactor vessel 14. The catalyst bed 164 constitutes the uppermost catalyst bed and the catalyst bed 172 constitutes the lowermost catalyst bed, with catalyst beds 166, 168, and 170 being intermediate catalyst beds positioned between the catalyst beds 164 and 172.

Each catalyst bed 164, 166, 168, 170, 172 has an upstream face 174 and an opposite downstream face 176 that are parallel to one another and oriented perpendicular to the longitudinal axis 16 of the reactor 160. In other embodiments, the catalyst beds 164, 166, 168, 170, 172 and their upstream and downstream faces 174, 176 are parallel to one another but non-perpendicular to the longitudinal axis 16.

Each of the catalyst beds 124, 126, 128, 130, 132, and 134 is configured so that at all or a portion of the outer side edges 178 of each catalyst bed are spaced radially inward from the interior surface of the reactor vessel wall 14 to define a longitudinal upstream flow space 180 along the length or a portion of the length of the reactor vessel 12. In the embodiment shown, the outer perimeter 178 is formed as a circle. In other embodiments, the catalyst beds may have an oval or non-circular outer edge or perimeter 178. In yet other embodiments, the catalyst beds may be configured into a partial or truncated circular shape having portions that are the same diameter as the vessel 12, the partial circles being truncated along one or both sides to leave a segment open for flow between the catalyst bed and the vessel wall.

Each catalyst bed 164, 166, 168, 170, 172 may have fluid impermeable enclosure structures 182 that connect the downstream side of each catalyst bed 164, 166, 168, 170 to central openings 184 formed in the next lower adjacent catalyst bed. In other embodiments, all or portions of the structures 182 may be formed from a layer of catalyst material so that the structure 182 forms a portion of the catalyst beds. As shown, the closure structure 182 includes a lower conduit 186 that extends to and is in fluid communication with the reactor outlet 22.

In an alternate arrangement, the lowermost catalyst bed 172 may have a side edge or perimeter 178 that extends radially outward so that it contacts or engages the cylindrical reactor wall 14 around its entire circumference. In such case, the enclosure structure 182 of the lowermost catalyst bed 172 and conduit 186 may be eliminated.

The catalyst bed assembly 162 formed by the catalyst beds 164, 166, 168, 170, 172, the closure structures 182, the conduit 186 or the lowermost catalyst bed 172 with the side edges 178 engaging the cylindrical reactor wall 14 divide the interior space 18 into an upstream zone 188 and a downstream zone 190. The upper face 174 of each of the catalyst beds 164, 166, 168, 170, 172 forms an upstream face and the lower face 176 of each catalyst bed forms a downstream face.

In use, the reactor 160 may be operated similarly to those reactors previously described as an AATR at conditions necessary to carry out the methane oxidative coupling reactions described herein. The feed gas mixture 116 from reactor inlet 20 flows as a flowing mixture comprising methane an oxygen gas within the upstream zone 188 of the interior of the reactor vessel 12. The feed gas mixture fills the entire volume of the upstream zone 188 of the reactor vessel 12, with the feed gas mixture flowing through the thickness of the catalyst beds 164, 166, 168, 170, 172 from the upstream face 174 to the downstream face 176 to the downstream zone 190. Portions or flows of the feed gas mixture pass only once through any catalyst bed to the downstream zone 190 without passing through any other catalyst bed.

As the flowing mixture flows through the OCM catalyst of the catalyst beds, the methane and oxygen of the flowing mixture reacts to form methane oxidative coupling reaction products, such as ethane and ethylene, that flow as effluent from the downstream face 176 of the catalyst beds to the downstream zone 190. The reaction products from downstream zone 190 are then discharged and removed from the reactor 160 through reactor outlet 22.

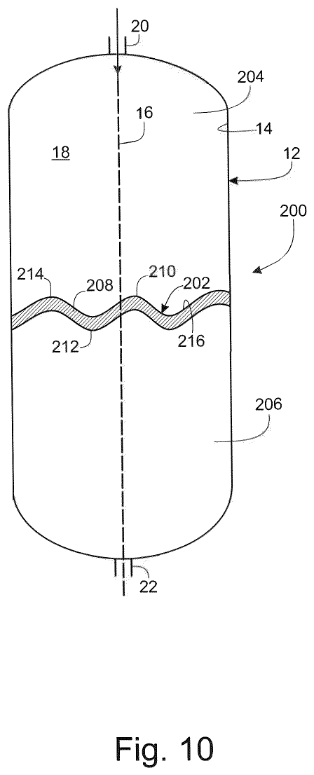

As shown in FIG. 10, a further embodiment of a reactor 200 employing a novel catalyst bed is shown. The reactor 200 includes a reactor vessel 12 having a reactor wall 14. A central longitudinal axis 16 extends the length of the reactor vessel 12. The reactor vessel 12 defines an interior 18 of the reactor 200. One or more inlets 20 of the reactor vessel 12 are provided for introducing one or more flowing feed gases 116 that comprise methane and oxygen gas into the interior of the reactor vessel 12. The reactor 200 is configured as an adiabatic reactor (or near-adiabatic) to prevent or reduce heat transfer or loss through the walls of the reactor vessel 12. Typically, the reactor 200 will be oriented for vertical flow, with the inlet 20 being located on the top or bottom of the reactor vessel 12. In the embodiment shown, the inlet 20 is located at the top of the reactor vessel 12 so that fluid flow is directed downward through the reactor 200.

The reactor 200 has a catalyst bed assembly 202 that divides the interior space 18 of the reactor vessel 12 into an upstream zone 204, which is in fluid communication with the inlet 20, and a downstream zone 206, which is in fluid communication with the reactor outlet 22.

The reactor 200 differs from those previously described in that the catalyst bed assembly 202 has a catalyst bed 208 that has a meandering, undulating, wavy or otherwise uneven surface with multiple peaks 214 and valleys 216 to thus increase the total area of the catalyst bed. The uneven surfaces may be arcuate or angular or a combination of arcuate and angular. The upstream face 210 and opposite downstream face 212 of the catalyst bed 208 have similar configurations and are spaced apart an equal distance over the entire extent of the catalyst bed 208 so that the height or thickness of the catalyst bed 208 is the same or substantially the same at any point of the catalyst bed. As shown, the sides or perimeter of the catalyst bed 208 extends to the interior of the reactor vessel wall 14 to close off the vessel 12.

It should be apparent, that two or more spaced apart catalyst beds, such as the catalyst bed 208, with meandering, undulating, wavy or otherwise uneven surfaces could also be used in the same reactor, such as with the multiple catalyst beds of the embodiment of FIG. 9.

The operation of the reactor 200 is similar to the operation of the reactors previously described and may be operated as an AATR.

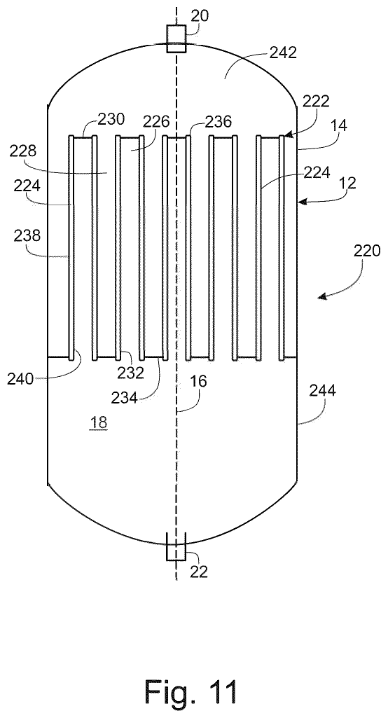

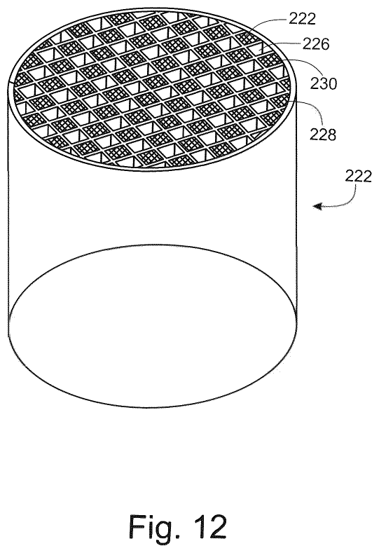

Referring to FIGS. 11-13, a further embodiment of a reactor 220 is shown. In the reactor 220 the catalyst bed assembly 222 has a honeycomb-type structure and is formed by longitudinally extending porous channel walls 224 that define longitudinally extending cell channels 226, 228. As can be seen more readily in FIG. 12, the channel walls 224 intersect in a lattice pattern. Closures or plugs 230 are provided at the upper or upstream end of the cell channels 226 with the bottom or downstream end 232 of the channel 226 being open. Closures or plugs 234 are provided at the lower or downstream end of the cell channels 228, with the upper or upstream end 236 of the cell channels 228 being open. As can be seen in FIG. 12, the cell channels 226, 228 are arranged in an alternating or checkerboard arrangement.

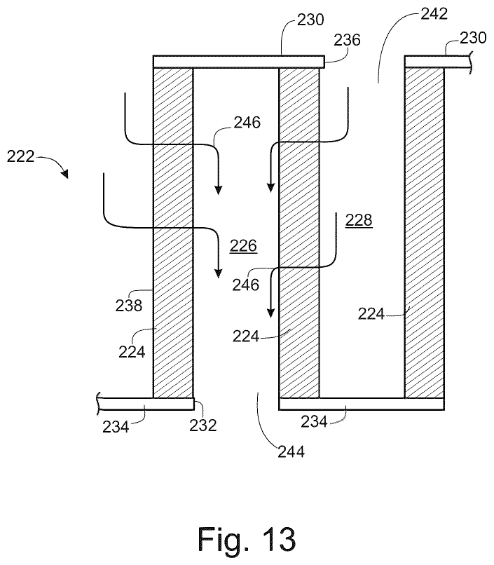

FIG. 13 shows enlarged cell channels 226, 228 with the channel walls 224 shown in cross section. The channel walls 224 are formed from a porous material that may be a porous OCM catalyst material or a porous non-OCM catalytic material that is coated with an OCM catalyst material or has pore channels containing an OCM catalyst material, such as has been described previously. Other configurations for the channel walls 224 incorporating an OCM catalyst material may also be used. The amount of OCM catalyst material of the channel walls 224 is that sufficient to carry out the OCM reaction, as described herein.

The catalyst material of the channel walls 224 forms the catalyst bed or beds of the catalyst bed assembly 222. As shown, the exposed face 238 within channel 228 of the catalyst bed 240 constitutes the upstream face and exposed face 240 within channel 226 of the catalyst bed 240 forms the downstream face.

The catalyst bed assembly 222 is disposed within the interior 18 of the reactor vessel 12 so that the interior 18 is divided into an upstream zone 242, which is in fluid communication with the reactor inlet 20, and a downstream zone 250, which is in fluid communication with the reactor outlet 22. The cell channels 228 constitute part of the upstream zone 242, while the cell channels 226 constitute part of the downstream zone 244.

The reactor 220 may be operated as an AATR at conditions necessary to carry out the methane oxidative coupling reactions described herein. The feed gas mixture 116 from reactor inlet 20 flows as a flowing mixture comprising methane and oxygen gas within the upstream zone 242 through the cell channels 228, which are open or unplugged at the upper or upstream ends 236. As shown by the arrows 246, the gas mixture passes through the catalytic channel walls 224 from the upstream face 238 to the downstream face 240. The effluent from downstream face 240 passes into cell channel 226, which is open or unplugged at the downstream end 232. As the flowing mixture flows through the OCM catalyst of the channel walls 224, the methane and oxygen of the flowing mixture reacts to form methane oxidative coupling reaction products. The reaction products are then removed from the downstream zone 244 through reactor outlet 22.

Each of the reactors described and shown may be configured to give selected dimensionless Peclet numbers based upon the flow through the reactor. The feed gas mixture is delivered to the reactor to provide a flowing gas mixture with a high enough velocity to provide a space time that is 500 milliseconds (ms) or less. In particular embodiments, the flowing mixture flow provides a space time of 500 ms, 400 ms, 300 ms, 200 ms, 100 ms, 50 ms, 25 ms, or 10 ms or less.

Based upon such flow rates to provide the selected space time during the autothermal reaction, the dimensionless Peclet (Pe) numbers provide a means for configuring the catalyst bed containing the OCM catalyst. The Pe numbers provide a means for determining the optimal catalyst bed depth or thickness. This can be achieved by calculating the three dimensionless Peclet numbers, i.e., the Heat Peclet Number (Peₕ), the Mass Peclet Number (Peₘ), and the Transverse Peclet Number (P).

The Heat Peclet Number (Peₕ) is defined in Equation (5) below:

$${Pe}_{h} = \frac{{uLC}_{pv}}{{k}_{b , eff}} \qquad ( 5 )$$

where, u is the superficial gas velocity, L is the catalyst bed depth or thickness, Cₚᵥ is the volumetric specific heat of the reaction mixture, and k_{b,eff} is the effective bed thermal conductivity. Peₕ is the ratio of thermal conduction time in the axial direction to the convection time within the catalyst bed and is an indication of thermal backmixing. In configuring the OCM catalyst beds of the reactors, a catalyst bed for the selected catalyst bed height or thickness and selected operating conditions, such as a space time of 500 ms or less, when subjected to the flowing gas mixture provides a Peₕ of from 5 or less. In certain embodiments, the catalyst bed under the selected operating conditions when subjected to the flowing gas mixture provides a Peₕ of from 5, 4, 3, 2, or 1 or less, more particularly from 2 or 1 or less.

The Mass Peclet Number (Peₘ) is defined in Equation (6) below:

$${Pe}_{m} = \frac{uL}{{D}_{m , eff}} \qquad ( 6 )$$

where, D_{m,eff} is the effective axial mass dispersion coefficient. Peₘ is the ratio of mass dispersion time in the axial direction to the convection time and is an indication of mass backmixing. In configuring the OCM catalyst beds of the reactors, a catalyst bed for the selected catalyst bed height or thickness and selected operating conditions when subjected to the flowing mixture provides a Peₘ of from 2 or more. In certain embodiments, the catalyst bed under the selected operating conditions provides a Peₘ of from 2, 3, 4 5, 6, 7, 8, 9, or 10 or more, more particularly from 5, 6, 7, 8, 9, or 10 or more.

The Transverse Peclet Number (P) is defined in Equation (7) below:

$$P = \frac{u}{{k}_{c} {a}_{v} L} \qquad ( 7 )$$

where, k_{c} is the local mass transfer coefficient and a, is the specific surface area of the catalyst (external catalyst surface area per unit volume of bed). P is the ratio of external mass transfer time (from the flow to the catalyst surface) to the convection time. In configuring the OCM catalyst bed of the reactor, a catalyst bed for the selected catalyst bed length or thickness and selected operating conditions when subjected to the flowing mixture provides a P of from 1 or less. In certain embodiments, the catalyst bed under the selected operating conditions provides a P of from 1, 0.9, 0.8, 0.7, 0.6, 0.5, 0.4, 0.3, 0.2, or 0.1 or less, more particularly from 0.3, 0.2, or 0.1 or less. Reactors with OCM catalyst beds that are operated in the autothermal state at high gas flow rates to provide a low space time of from 500 ms or less and that meet the specified Peclet number parameters presented above can be used in an adiabatic autothermal operation in which the feed gas mixture can be used as the coolant and which provides increased yields of OCM reaction products of C₂+ hydrocarbons and high volumetric productivity than with conventional OCM reactors.

The gaseous feedstocks used with the reactors and method include a hydrocarbon feed gas, particularly methane, and oxygen gas. The hydrocarbon feed gas may include methane, natural gas, light-alkane gases (e.g., C₂-C₆), etc. All or a majority portion of the hydrocarbon feed gas may comprise methane in certain embodiments. The hydrocarbon feed gas may be a methane-containing gas that may be a pure methane gas or may be methane gas source containing other gases. In certain instance, the hydrocarbon feed gas may be predominantly methane (i.e., >50 mol %) or entirely methane. In particular embodiments, the hydrocarbon feed gas may be composed of natural gas, which may have a methane content of from 85 mol % to 97 mol % or more, or other hydrocarbon-rich gases. In some cases, the hydrocarbon feed gas may be pretreated to remove undesirable components, such as sulfur-containing compounds and the like. The gaseous feedstocks also include oxygen-containing gas, which may be air, oxygen-enriched air, or pure oxygen gas. In particular embodiments, the oxygen-containing gas is pure oxygen. The reactant gas mixture may contain other gases, provided such gases do not negatively affect the reaction. These can include nitrogen (N₂), carbon dioxide (CO₂), hydrogen (H₂), etc.

The OCM reaction may be ignited in a manner that avoids transient states at which the temperatures would destroy or damage the OCM catalyst of the catalyst bed. During the reaction startup, the feed gas mixture may have a selected HC/O₂ or CH₄/O₂ molar ratio, which may range from 3:1 to 40:1, is initially heated to a temperature of at least 400° C., more particularly from 400° C. to 750° C. The heated feed gas mixture is introduced into the OCM reactor so that the catalyst bed is also heated and the OCM reaction commences. The feed gas mixture may be introduced to provide a space time of from 0.1 ms to 1000 ms. Upon ignition, the temperature and/or HC/O₂ or CH₄/O₂ molar ratio can be incrementally reduced over a startup period. Additionally, the flow rate may be adjusted to provide a shorter space time. Once a selected operating temperature is achieved, the OCM reaction may remain ignited and the reactor or reaction zone can be maintained in an autothermal state.

Once the OCM reaction is in an ignited state, the reaction may be made to operate continuously in an autothermal state by supplying feed gas for the oxidative coupling reaction to the reactor at a rate and at a low enough temperature to compensate for the heat of reaction generated in the reactor. In this way, the feed gas serves as a coolant as the reactor is heated to a higher temperature by the heat generated by the oxidative coupling reaction in the reactor. Once the reactor reaches the autothermal state, the feed gas mixture to the reactor may comprise a hydrocarbon gas (HC) or methane-containing gas, which may contain at least some portion of methane (CH₄), and oxygen gas (O₂). The HC/O₂ or CH₄/O₂ molar ratio of the feed gas mixture may range from 2.5:1 to 10:1, more particularly from 3:1 to 9:1.

The temperature of the feed gas mixture during the autothermal state, which is a HC-containing or methane-containing feedstock along with an oxygen-gas-containing feedstock, is introduced into the reactor at a temperature of from −100° C. to 300° C. In particular embodiments, the temperature of the mixed gas feedstock introduced into the reactor ranges from −20° C. to 150° C., more particularly from −20° C., −10° C., or 0° C. to 50° C., 100° C., or 150° C. The cooler feedstock gas mixture introduced into the reactor and the high space velocity facilitates maintaining the catalyst bed temperature at the desired temperature during the autothermal state even while the OCM generates a significant amount of heat during the reaction.

The OCM reactors are operated in the ignited or autothermal state to provide a catalyst bed temperature of from 500° C. to 1000° C. In particular embodiments, the reactors are operated to provide a catalyst bed temperature of from 800° C. to 950° C. in the ignited or autothermal state. The reactors may be operated at a pressure of from 0.1 MPa to 1 MPa, more particularly from 0.1 MPa to 0.5 MPa in the autothermal state.

The products produced from the OCM reaction include ethane, ethylene, as well as other C₂+ hydrocarbon products, along with carbon oxides like CO and CO₂.

As can be seen from the various reactors and catalyst bed configurations described above, the volumetric productivity of reactors in OCM reactions can be effectively increased by significantly increasing the surface area of upstream face of the catalyst beds to the incoming reactor feed using the various different configurations shown and described. In this way, reactors with conventional dimensions can be used for OCM reactions without increasing their numbers or increasing their size to increase their volumetric productivity.

While the disclosure has been shown in some of its forms, it should be apparent to those skilled in the art that it is not so limited, but is susceptible to various changes and modifications without departing from the scope of the disclosure based on experimental data or other optimizations considering the overall economics of the process. Accordingly, it is appropriate that the appended claims be construed broadly and in a manner consistent with the scope of the disclosure.

1. An oxidative methane coupling (OCM) reactor comprising:
a reactor vessel that defines a reactor vessel interior, wherein the reactor vessel comprises opposite ends and a central longitudinal axis that extends between the opposite ends;
a reactor inlet for introducing one or more flowing feed gases comprising methane and oxygen gas into the interior of the reactor vessel;
a catalyst bed assembly positioned within the reactor vessel interior having at least one catalyst bed containing a layer of OCM catalyst,
wherein (i) the catalyst bed assembly divides the reactor vessel interior into an upstream zone and a downstream zone, (ii) the at least one catalyst bed of the catalyst bed assembly having an upstream face for receiving the one or more flowing feed gases as a flowing mixture from the upstream zone, (iii) the upstream face of the at least one catalyst bed of the catalyst bed assembly is configured to have a total area that exceeds the largest transverse cross-sectional area of the interior of the reactor vessel that is perpendicular to the central longitudinal axis of the reactor vessel, and (iv) the at least one catalyst bed assembly is configured so that any portion of the flowing mixture passes from the upstream zone to the downstream zone through only a single catalyst bed of the catalyst bed assembly; and
a reactor outlet located downstream from the catalyst bed assembly that is in fluid communication with the downstream zone for removing reaction products from the reactor.

2. The reactor of claim 1, wherein the upstream face is non-perpendicular to the central longitudinal axis of the reactor vessel.

3. The reactor of claim 1, wherein the upstream face or a portion of the upstream face of the at least one catalyst bed is configured as at least one of a cone, a partial cone, a cylinder, a partial cylinder, a cube, a partial cube, a cuboid, a partial cuboid, a sphere, a partial sphere, a spheroid, a partial spheroid, a pyramid, a partial pyramid, a plane that is non-perpendicular to the central longitudinal axis of the reactor vessel, and a non-planar face.

4. The reactor of claim 3, wherein the upstream face of the at least one catalyst bed is configured as a cylinder or partial cylinder.

5. The reactor of claim 1, wherein the catalyst bed assembly comprises at least two catalyst beds having upstream faces configured as two or more planes that are parallel to one another.

6. The reactor of claim 5, wherein the two or more planes are perpendicular to the central longitudinal axis of the reactor vessel.

7. The reactor of claim 3, wherein the upstream face is configured as a cone or partial cone.

8. The reactor of claim 1, wherein the catalyst bed assembly comprises at least two catalyst beds that are spaced apart along the longitudinal axis of the reactor vessel.

9. The reactor of claim 1, wherein the layer of OCM catalyst has a thickness of 50 mm or less.

10. The reactor of claim 1, wherein the total area of the upstream face of the at least one catalyst bed exceeds the largest transverse cross-sectional area of the interior of the reactor vessel that is perpendicular to the central longitudinal axis of the reactor vessel by 50% or more.

11. The reactor of claim 1, wherein the catalyst bed assembly comprises only one catalyst bed.

12. The reactor of claim 1, wherein the at least one catalyst bed is a contiguous catalyst bed that has a symmetrical configuration about the central longitudinal axis of the reactor.

13. The reactor of claim 1, wherein (i) the at least one catalyst bed is configured with porous channel walls that define longitudinally extending cell channels, (ii) the layer of OCM catalyst being positioned along at least a portion of the cell channels, (iii) a portion of the cell channels is closed at an upstream end with the downstream end being open to form an outlet of the cell channels, and (iv) wherein the remainder of the cell channels are closed at the downstream end and open at the upstream end to form an inlet of the cell channels.

14. The reactor of claim 1, wherein at least one catalyst bed is configured to have a heat Peclet number (Peₕ) of from 5 or less.

15. A method of carrying out autothermal oxidative coupling of methane (OCM) comprising:
introducing one or more flowing feed gases comprising methane and oxygen gas into an adiabatic reactor as a flowing mixture, the reactor comprising:
a reactor vessel that defines a reactor vessel interior, wherein the reactor vessel comprises opposite ends and a central longitudinal axis that extends between the opposite ends;
a reactor inlet for introducing one or more flowing feed gases comprising methane and oxygen gas into the interior of the reactor vessel;
a catalyst bed assembly positioned within the reactor vessel interior having at least one catalyst bed containing a layer of OCM catalyst, wherein (i) the catalyst bed assembly divides the reactor vessel interior into an upstream zone and a downstream zone, (ii) the at least one catalyst bed of the catalyst bed assembly having an upstream face for receiving the flowing mixture from the upstream zone, and (iii) the upstream face of the at least one catalyst bed of the catalyst bed assembly is configured to have a total area that exceeds the largest transverse cross-sectional area of the interior of the reactor vessel that is perpendicular to the central longitudinal axis of the reactor vessel; and
a reactor outlet located downstream from the catalyst bed assembly that is in fluid communication with the downstream zone for removing reaction products from the reactor;
passing the flowing mixture from the upstream zone to the downstream zone so that any portion of the flowing mixture flows through only a single catalyst bed of the catalyst bed assembly, wherein methane and oxygen gas of the flowing mixture contact the OCM catalyst of the at least one catalyst bed to undergo an auto-thermal OCM reaction to form methane oxidative coupling reaction products that are received in the downstream zone; and
removing the reaction products from the downstream zone through the reactor outlet.

16. The method of claim 15, wherein at least one catalyst bed is configured to have a heat Peclet number (Peₕ) of from 5 or less.

17. The method of claim 15, wherein the OCM catalyst has a uniform thickness of 50 mm or less.

18. The reactor of claim 1, wherein the OCM reactor comprises an adiabatic reactor.

19. The reactor of claim 1, wherein the OCM catalyst has a uniform thickness.

20. The reactor of claim 19, wherein the thickness of the OCM catalyst is 50 mm or less.
